(19) United States
(12) Patent Application Publication
(10) Pub. No.: US 2010/0105127 A1
(43) Pub. Date: Apr. 29, 2010

(54) SYSTEMS AND METHODS FOR GENERATING RESOURCES USING WASTES

(75) Inventor: Martin A. Ginsburg, Raleigh, NC (US)

Correspondence Address: OLIVE LAW GROUP, PLLC, 130 EDINBURGH SOUTH DRIVE, SUITE 107, CARY, NC, 27511, US

(73) Assignee: MarGin Consulting, LLC, Raleigh, NC (US)

(21) Appl. No.: 12/576,293

(22) Filed: Oct. 9, 2009

Related U.S. Application Data
(60) Provisional application No. 61/108,208, filed on Oct. 24, 2008.
(60) Provisional application No. 61/228,992, filed on Jul. 28, 2009.

(51) Int. Cl. C02F 3/00; C12M 1/00

(52) U.S. Cl. 435/262; 435/290.1

(57) ABSTRACT

Systems and methods for generating resources using municipal solid waste are disclosed herein. According to one aspect, a method includes receiving wastes. The wastes can be separated into different portions for different downstream processes. Further, the wastes can be treated in an anaerobic digestion process for producing biogas. A gasification process can be applied to the wastes for producing synthesis gas. Further, an algal growth system can be applied for sequestering system-produced carbon dioxide (CO₂).

### CROSS REFERENCE TO RELATED APPLICATIONS

This application claims the benefit of U.S. provisional patent application No. 61/108,208, filed Oct. 24, 2008, and U.S. provisional patent application No. 61/228,992, filed Jul. 28, 2009, the contents of which are incorporated herein in their entireties.

### TECHNICAL FIELD

The subject matter disclosed herein relates to the processing of wastes. Particularly, the subject matter disclosed herein relates to generating resources using wastes.

### BACKGROUND

As nations become more industrialized and the world more populated, there is a constant increase both in the demand for electricity and in the generation of waste. One of the largest problems faced by municipalities, and by society in general, is the increase in the amount of municipal solid waste and other wastes generated each year. For instance, almost 350 million tons of waste is produced each year in North America alone. A study by the United States Environmental Protection Agency (EPA) has revealed that there has been a 42% increase in recycling and composting from 1988 to 1995. Surprisingly, during this same period, the quantity of municipal solid waste still increased. Certified landfill capacity is decreasing and other sites require clean-up. New options of waste management to replace traditional methods, e.g., open dumping, landfills and composting, are needed in order to manage the millions of tons of municipal solid waste that are produced each year.

One way to alleviate the problems associated with waste is to recover the potential energy otherwise lost as waste. Many current waste-to-energy systems are “through-systems” in which the waste is combined with fuel as required and burned. In a typical 50 megawatt plant, approximately 5000 tons of waste can be burnt per day. There are, however, several disadvantages to this system. For instance, 50% to 60% of the waste processed in such systems is transformed into incompletely burned bottom ash. In addition, fly ash is produced, which is classified as hazardous waste. Bottom ash must be shipped to a landfill site, and the fly ash requires a hazardous waste facility.

Electrical energy today is typically generated by power plants that burn fossil fuels such as coal, natural gas or heavy diesel oil to generate electricity. Such plants, however, also generate significant air pollution. Nuclear power plants produce electricity more cleanly, but they are being phased out worldwide due to popular concern over their perceived risks and the radioactive nature of the waste they generate. In view of the increasing costs and dwindling supply of fossil fuels, many countries are recognizing and encouraging the production of electrical energy from renewable sources of fuel, such as wind, solar, hydro and waste/biomass.

Waste (including municipal solid wastes (MSW), industrial waste, toxic waste, bio-hazardous/hospital wastes, agricultural wastes (especially animal wastes), forest/crop/urban/industrial wood residues, organic materials from municipal and other wastewaters, municipal storm water runoff, and coal ash and fines, or other non-nuclear waste) is currently being dumped into polluting landfills, discharged into regional surface aquifers, or being burned in common incinerators, creating emissions of pollutants, including carcinogenic materials such as semi-volatile organic compounds (SVOCs)—dioxins, furans, etc.—that are products of low temperature combustion.

Landfills are becoming full, and the availability of new sites near heavily populated areas is limited worldwide. Additionally, the continued pollution of surface and ground water by hazardous leachate and the discharge of inadequately treated wastewater as well as health concerns caused by malodor, rodents and fumes, have rendered landfills undesirable as well as introducing pollutants into aquifers. These issues and others have resulted in the development of the “not in my backyard” (“NIMBY ”) syndrome in most populations. For these reasons, the European Union is working to force closure of all landfills and mandating that existing landfills meet new, more stringent leachate and pollution control standards, thus increasing the costs of landfills markedly. Unfortunately, to date, little has been done to address the discharge of inadequately treated wastewater and storm waters into aquifers.

Incinerators also have been closed down or banned in many countries because of hazardous air emissions and resulting ash production. As a result of the low temperature combustion that takes place in these incinerators, hydrocarbon chains are not completely severed and are released into the atmosphere as SVOCs, which are known carcinogens and are passed through to humans via the food chain, for example, as dioxins that are deposited on grass and eaten by cattle and end up in milk sold to humans. The fixed carbons in the waste also are untouched by the low temperature incineration process and end up as bottom ash and fly ash. This ash makes up almost 25% of the waste and is considered hazardous due to its leachability once land-filled. Many countries are now prohibiting the direct landfill of ash.

Therefore, there exists a need both for a source of readily renewable electrical energy produced from multiple waste streams, and for a method to access this resource in a most efficient manner without a concomitant production of pollution. A number of processes have been developed claiming a “closed loop” system. This claim disregards the fact that there is energy inputted to the system. The claim seems based upon a reduction of waste generated by the processes involved. There remain, however, several disadvantages to, or problems not solved by, the processes previously developed and thus far deployed in that they fail to make the most benefit of materials inputted to their respective processes, as well as failing to recover available energy to the limit of the current state of the art leading to reduced efficiency and increased cost.

Basic power generation technologies are generally grouped according to the energy source used to produce electricity. Fossil fuels such as coal, natural gas and oil are used to produce steam which is expanded through a steam turbine which, in turn, drives a generator thereby producing electric power. Fuels can also be combusted as in a gas turbine, where the primary energy source is hot gas which again expands and drives a generator. Nuclear power also uses a steam turbine-generator to convert steam produced by a nuclear reactor into power. In the case of geothermal power generation, steam naturally produced by the earth is extracted and processed to an extent, for expansion again, in a steam turbine-generator, although at much lower temperatures and pressures than the aforementioned fossil fuels. While the efficiencies associated with the geothermal steam are much lower than that of the traditional fossil fuels, the steam is essentially free, after the installed cost of the delivery infrastructure, compared to the cost of fossil fuel necessary to produce like amounts of steam. Solar power has also been used to boil water for steam as in Solar One, a plant near Dagget, Calif. Such geothermal systems, just as fossil based oil and natural gas, leave voids in the Earth's crust whose implications in natural disasters, such as earthquake, has not been measured or estimated.

Technologies, such as hydroelectric generation, utilize the extraction of potential energy from water moved from higher elevations to lower elevations, using the rush of falling water through a “Francis” or “Kaplan” impulse turbine in order to turn a generator and produce electricity. There is no need to produce steam in such a system. The impinging force of the water acting on the water turbine provides the energy to be extracted.

While naturally occurring energy sources such as sunlight or water are “free,” they can vary in supply. In dry years, less hydroelectric generation is available. On cloudy days, less solar power can be generated. Where wind turbines are concerned, a mean wind velocity of at least 10 mph is required to justify installation, because if there is no wind, power is not produced. Similarly, geothermal fields finally expend their available steam, rendering the massive distribution system and generating equipment installed above the field useless. Utility companies and power associations have traditionally attempted to manage such systems: placing hydroelectric systems proximate to predictable watersheds and by building reservoirs; installing arrays of wind turbines in established zones of plentiful and predictable wind currents; building solar plants in desert locations, etc.

Today, in an effort to increase generation thermal efficiencies, technologies are sometimes combined. An example of such a combination is steam and gas turbine technology. In such a system, a gas turbine power plant is used to generate electricity, and concurrently, the exhaust gases, at nearly 950° F., are directed through a heat recovery boiler to produce steam which is then expanded through a traditional steam turbine-generator. This combination increases the overall thermal efficiency beyond that seen with either gas or steam technology separately. These combinations are typically not available in naturally occurring energy resources.

A gas turbine power plant can include, but not be limited to: a compressor for compressing air; and a combustor for burning fuel derived from system processes including at least one of gasification-derived synthesis gas, FT synthesis fuels, biogas, biodiesel, ethanol, or other carbonaceous fuel with compressed air from the compressor to produce combustion gas. The power plant also includes a turbine configured to be driven by the combustion gas. The power plant may also include: a generator configured to be driven by the turbine for producing electric power; a heat exchanger configured to apply exhaust heat to a Rankine Cycle system for electricity production; and a regenerative heat exchanger configured to heat the compressed air with the heat of exhaust gas of the turbine.

Efforts to find other renewable energy sources to reduce dependence on fossil fuels have spawned alternate fuels including the burning of agricultural waste such as wood chips, almond shells and rice hulls to generate power. Used tires, municipal solid waste in the form of a screened mass or refuse-derived fuel have also provided fuel for power generation. In the case of municipal solid waste, the fuel has been exploited in large part to reduce the amount of waste sent to landfills. The utilization of wastes in the generation of electric power is a very promising renewable energy technique.

What is needed, then, are ways to extend or augment the availability of renewable or natural resources beyond traditional system efficiency improvements, in order to prolong available energy resources and reduce the dependency on fossil fuels. Generally, it is desired to provide improved systems and methods for generating resources using wastes.

### SUMMARY

Systems and methods for generating resources using wastes are disclosed herein. According to one aspect, a method includes receiving wastes, which can include, but is not limited to, MSW, industrial wastes, recyclable materials, plastics, textiles, ferrous metals, non-ferrous metals, organic materials, electronics, industrial waste, bio-hazardous materials, medical materials, hospital wastes, municipal waste water, municipal storm water, residential wastes, commercial wastes, wood residues, animal wastes, and food processing wastes. The wastes can be separated into different portions for different downstream processes. Further, the organic constituents of the wastes can be treated in an anaerobic digestion process for producing biogas. A gasification process can be applied to the wastes for producing synthesis gas. Further, an algal growth system can be applied for sequestering system-produced carbon dioxide (CO₂). The synthesis gas can undergo a Fischer-Tropsch (FT) synthesis process for producing synthetic fuels or other valuable products.

According to an aspect, the wastes include recyclable and non-recyclable materials. The recyclable materials can be separated from the non-recyclable materials. Subsequently, the recyclable materials can be appropriately transported for recycling. A suitable gasification process can be applied to the recyclable materials for producing a synthesis gas. Further, the gasification process can be used to produce slag. In an example, the gasification process can utilize one of a fluidized bed, a plasma arc, or a plasma torch.

According to another aspect, the wastes can include organic materials, municipal wastewater and municipal storm water. The organic materials can be processed in an anaerobic digestion process.

Biogas resulting from an anaerobic digestion process can include H₂S, CO₂, and methane. Some or all of the H₂S can be removed from the biogas. An algal growth process can be applied to the biogas to remove and sequester at least a portion of the CO₂ therein to increase methane gas concentrations. The methane gas can be utilized as a fuel source to heat process water for treatment and steam production. The heat and steam can be employed for electricity production. The heat can be employed to process water for pasteurization. In addition, heat energy can be recovered for other system processes. The heat can also be employed for heating organic waste preparatory to the anaerobic digestion process.

According to yet another aspect, a method in accordance with the subject matter disclosed herein can utilize produced synthesis gas in a power production system. The synthesis gas may be used in an integrated gasification combined cycle (IGCC) power production system for producing electricity. Further, carbon dioxide resulting from the power production system can be captured. The carbon dioxide can then be applied to an algal system process for CO₂ sequestration.

According to still yet another aspect, a tipping facility can be used for separating wastes. Unfiltered air within the tipping facility can be used as air intake in a combustion system.

According to another aspect, the gasification process can be used to produce heat. In addition, one or both of an Organic Rankine Cycle (ORC) system and Heat Recovery Steam Generator (HSRG) can be provided.

According to another aspect, the method disclosed herein can utilize a gas turbine power plant. The power plant can include a compressor for compressing air. Further, the power plant can include a combustor for burning fuel derived from system processes including at least one of gasification-derived synthesis gas, FT synthesis fuels, biogas, biodiesel, ethanol, or other carbonaceous fuel with compressed air from the compressor to produce combustion gas. A turbine can be configured to be driven by the combustion gas. A generator can be configured to be driven by the turbine for producing electric power. A heat exchanger can be configured to apply exhaust heat to a Rankine Cycle system for additional electricity production. A regenerative heat exchanger can be configured to heat the compressed air with the heat of exhaust gas of the turbine or Rankine cycle system.

According to yet another aspect, a solar power system can be utilized for producing electricity to implement one of more of the steps of a method in accordance with the subject matter disclosed herein. Exemplary systems include concentrated solar power systems, photovoltaic systems, and other such systems known to those of skill in the art.

According to yet another aspect, waterborne waste can be sequestered from any of the steps of a method in accordance with the subject matter disclosed herein. The water separated from waterborne waste can be used for producing potable water.

According to still yet another aspect, wastes received in accordance with the disclosed subject matter can include fibrous organic waste, grit material, and heavy waste material. The fibrous organic waste, grit material, and heavy waste material can fully or at least partially be separated from each other. The fibrous organic waste can be prepared for anaerobic digestion. The grit material can be transported for one or both of recycling and disposal. Portions of the heavy waste material can be separated into recyclable ferrous metals and non-ferrous metals. Gasification may be applied to any grit material and/or heavy waste material after suitable preprocessing.

According to another aspect, a gasification process can be applied to algae harvested from the algal growth system for producing additional synthesis gas. The additional synthesis gas can be used in a power production system. Carbon dioxide resulting from the power production system can be captured and applied to the algal growth system for carbon dioxide sequestration.

According to yet another aspect, a gasification process can be applied to algae harvested from the algal growth system for producing additional synthesis gas. The additional synthesis gas can undergo Fischer-Tropsch synthesis (FTS) for the production of liquid fuels from synthesis gas. Fischer-Tropsch fuels can serve as a direct replacement for automotive, aviation or other fuels without requiring modifications of the use systems.

According to another aspect, olefins, paraffins, tail gas or other by-products or co-products of FTS can be returned to a gasification process to enhance its thermal efficiency.

According to yet another aspect, a transesterification/esterification process can be applied to lipids separated from harvested algae for producing biodiesel. The biodiesel can be used in a power production system. Carbon dioxide resulting from the power production system can be captured and applied to an algal system process for carbon dioxide sequestration.

According to another aspect, a liquefaction/fermentation/distillation process can be applied to the cellulosic materials separated from harvested algae produced in accordance with the disclosed subject matter for producing ethanol. The ethanol can be used in a power production system. Carbon dioxide resulting from the power production system can be captured and applied to the algal growth system for carbon dioxide sequestration.

According to another aspect, effluent from the anaerobic digestion process in accordance with the disclosed subject matter can be dewatered. Further, filtration and heat can be applied to recovered water from dewatering for producing potable water.

According to yet another aspect, effluent from the anaerobic digestion process in accordance with the subject matter disclosed herein can be dewatered for producing solids. The solids can be dried. Further, a gasification process can be applied to the dried solids for producing a synthesis gas.

According to yet another aspect, effluent from the anaerobic digestion process in accordance with the subject matter disclosed herein can be dewatered for producing solids. Vermiculture can be applied to the dried solids for producing a slow-release soil amendment.

According to yet another aspect, effluent from the anaerobic digestion process in accordance with the subject matter disclosed herein can be dewatered for capture of high nutrient content water. Further high nutrient water extracted from anaerobic digester effluent can be pasteurized and suitably prepared for direct land application.

According to another aspect, a system in accordance with the disclosed subject matter can include any suitable components for implementing the above described methods.

It is a fundamental property of the presently disclosed subject matter that waste is a resource out of place and need only find an appropriate path through a treatment process to become a resource rather than additional discard. Utilization of these heretofore underutilized and ignored resources can lead to reduced production costs and increased profit potential while still permitting lower cost to consumers through savings realized in capture, manufacturing, and production processes.

Existing processes and systems have addressed a number of needs across a variety of arts yet have failed to address these concerns holistically. By taking an approach that views these problems as interconnected and realizing that only a solution that addresses these concerns as constituent parts of a whole, the presently disclosed subject matter seeks to correct, rather than repair, these problems. It is through the application of a combination of existing technologies that the presently disclosed subject matter eliminates waste from a variety of sources while producing a plethora of valuable products for use worldwide. The potential end-products of this process include, but are not limited to, production of electricity, Fischer-Tropsch derived fuels to replace gasoline/diesel/aviation fuels, industrial chemicals, pharmaceutical/nutraceutical products, separated precious metals, separated base metals, algae derived biodiesel/ethanol/aviation fuels, carbon credits through the capture of all system recycled and generated carbon dioxide, pipeline grade methane gas, potable equivalent water, soil amendment, animal bedding, animal feedstock, and oxygen rich gas exhausted from an enclosed algal growth system.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not limited to implementations that solve any or all disadvantages noted in any part of this disclosure.

### BRIEF DESCRIPTION OF THE DRAWINGS

The foregoing summary, as well as the following detailed description of preferred embodiments, is better understood when read in conjunction with the appended drawings. For the purposes of illustration, there is shown in the drawings exemplary embodiments; however, the presently disclosed subject matter is not limited to the specific methods and instrumentalities disclosed. In the drawings:

FIG. 1 illustrates an exemplary process for generating resources using wastes in accordance with an embodiment of the subject matter disclosed herein; and

FIGS. 2A-2I illustrate a block diagram of an exemplary system for generating resources using wastes according to an embodiment of the subject matter disclosed herein.

### DETAILED DESCRIPTION

Systems and methods for generating resources using wastes are disclosed herein. The presently disclosed subject matter is described with specificity to meet statutory requirements. However, the description itself is not intended to limit the scope of this patent. Rather, the inventor has contemplated that the claimed subject matter might also be embodied in other ways, to include different steps or elements similar to the ones described in this document, in conjunction with other present or future technologies. Moreover, although the term “step” may be used herein to connote different aspects of methods employed, the term should not be interpreted as implying any particular order among or between various steps herein disclosed unless and except when the order of individual steps is explicitly described.

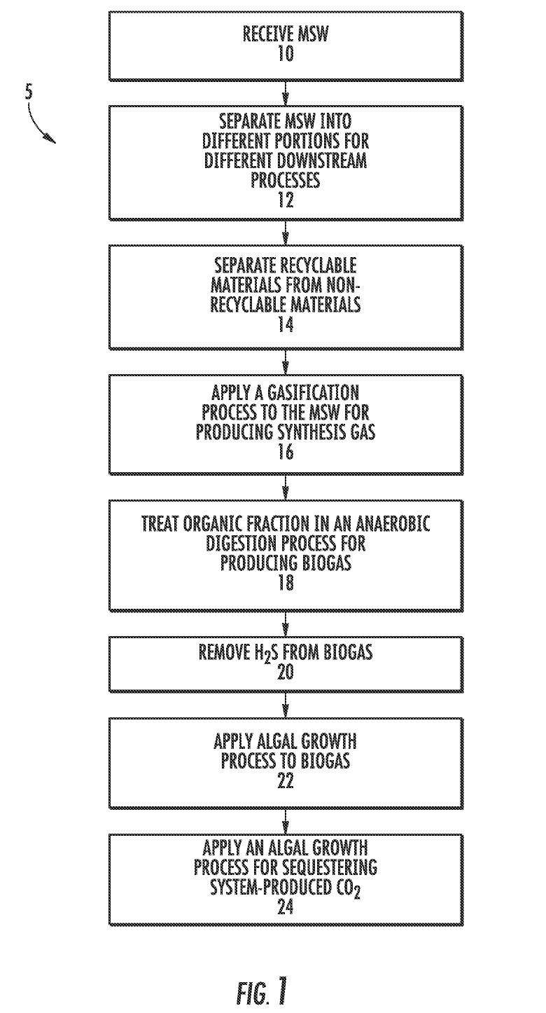

FIG. 1 illustrates an exemplary process 5 for generating resources using wastes in accordance with an embodiment of the subject matter disclosed herein. Referring to FIG. 1, wastes can be received at a waste treatment facility (step 10). The wastes can include, for example, recyclable materials, plastics, textiles, ferrous metals, non-ferrous metals, organic materials, electronics, industrial waste, bio-hazardous materials, medical materials, hospital wastes, municipal waste water, municipal storm water, residential wastes, commercial wastes, wood residues, animal wastes, food processing wastes, and the like. Waste is not, as applied in the processes described herein, limited to curbside or bulk pick-up of residential waste as is generally accepted. By expanding the definition of wastes to include industrial wastes and bio-hazardous/medical/hospital wastes, the advantage to waste producers other than residential customers can be derived through a lower cost waste disposal option. These wastes, delivered separately to prevent cross-contamination of delivery vehicles, can be further separated on-site as a part of the processes described in further detail herein and be processed for elimination of disposable waste as well as production of valuable product output.

At the waste treatment facility, the wastes can be separated into different portions for downstream processes (step 12). These processes may occur sequentially or concurrently in an effort to eliminate waste while producing valuable products as process output. Each process can be expansive in scope and effort is made herein to disclose each stream individually with the understanding that the streams may be co-dependent as described herein. Wastes can be segregated using a secondary tipping facility and comingled with other waste constituents to permit concomitant processing of wastes and its constituents. As described in further detail herein, after anaerobic digestion, these wastes may be compressed with dried anaerobic digestion sludge infused to reduce entrapped air that might potentially interfere with the gasification process. Unfiltered air captured from the tipping facility may be used as air intake in a combustion system as described herein.

The wastes can be processed downstream from its receipt at the treatment facility using a number of different modalities, which may be dependent upon practices employed at a particular municipality. For example, recycling practices of an area serviced by the process disclosed herein can influence the application of a practice mode employed at a given site. At step 14, recyclable materials in the wastes can be separated from the non-recyclable materials. At least a portion of the recyclable materials may be loaded onto transport equipment, such as trucks, for transporting to another site for recycling as known to those of skill in the art. Alternatively or in combination with transporting some of the recyclable materials to other sites, gasification can be applied in a gasification system to a portion of the recyclable materials or other wastes for producing a synthesis gas (step 16).

A gasification system may be generally defined as an enclosed thermal device and associated gas cleaning system or systems that does not meet the definition of an incinerator or industrial furnace as known to those of skill in the art. A gasification system can limit oxygen concentrations in the enclosed thermal device to prevent the full oxidation of thermally-associated gaseous compounds. A gas cleanup system or systems designed to remove contaminants from the partially oxidized gas that do not contribute to its fuel value may also be employed. The system can transform inorganic feed materials into a molten, glass-like substance (referred to as “slag”) at temperatures above about 2000° F., and can produce a synthesis gas. Gasification of waste intake can be accomplished using any gasification technology known to those of skill in the art, such as, for example, fluidized bed technology, plasma arc technology, and plasma torch technology. Plasma gasification provides a wide application spectrum in that plasma gasification is capable of processing wastes of greater moisture content than that acceptable in a fluidized bed gasification process as well as having the advantage of limited to no formation of tars and other contaminants. This is a significant improvement over non-thermal or plasma assisted gasification processes in that the tars and other contaminants eventually become material suitable primarily for disposal with more limited alternative uses. The inclusion of industrial wastes in the input stream can, by nature of urban waste streams, include high carbon liquids in the form of used motor oil and lubricants, as well as fats, oils, and grease (FOG) from commercial food production and service operations. The inclusion of these wastes in a stream undergoing fluidized bed gasification can mitigate the negative impact of higher moisture content of the incoming wastes, however, plasma gasification can result in more complete dissociation of components leaving significantly less to no residual materials for disposal while increasing synthesis gas quality. Gasification produces a synthesis gas (syngas), also known as “producer gas,” comprised predominantly of hydrogen and carbon monoxide. This gas, when scrubbed to remove tars and other contaminants, can be used to operate a power production system. Exhaust gases can be captured from such power generation equipment and the CO₂ contained therein utilized in an algal growth system, as described in further detail herein. Heat from the exhaust of this process can be utilized as source heat as needed in other processes, as described in further detail herein.

Synthesis gas, created by the gasification of wastes, can be scrubbed using any suitable process known to those of skill in the art. Rapid cooling of these scrubbed gases permits their use in a number of processes. For example, this gas may be used in an Integrated Gasification Combined Cycle (IGCC) power production system for the direct production of electricity. Synthesis gas may also undergo an alternative treatment for the production of liquid fuels capable of use in the existing transportation/delivery/use infrastructure without having to overcome the burdens currently faced by biodiesel and ethanol.

Another process that can be employed is Fischer-Tropsch synthesis (FTS), which is a process for the production of liquid fuels from syngas. This process is currently employed in the production of liquid fuels from the gasification of natural gas or coal. Further, this process is suitable for use following the gasification of wastes and/or the organics remaining following anaerobic digestion of organic wastes input to this process, as described in more detail herein. Syngas captured from gasification can be scrubbed, as needed, and converted to liquid fuels capable of functioning as direct replacements for gasoline, diesel and aviation fuels that require no adaptations to the current transportation/distribution/delivery/use infrastructures. Liquid fuels generated by implementation of FTS potentially contain no sulfur and are cleaner burning than fossil derived liquid fuels, according a U.S. Environmental Protection Agency (EPA) “Success Story” publication dated March of 2002 and titled “Clean Alternative Fuels: Fischer-Tropsch”. Byproducts of FTS, including olefins and solid paraffin can be returned to a gasification process owing to their high hydrocarbon content.

At step 18, the wastes are treated in an anaerobic digestion process for producing biogas. In an example, this process can be applied to sewage, other waste water, or any suitable organic materials to produce biogas including, but not limited to, methane, carbon dioxide, and trace gases. The inclusion of wastes from concentrated animal feeding operations (CAFOs) as well as municipal storm water run-off as a diluent can present benefits readily identifiable by those of skill in the art. Solids separated from effluent can be further processed through a number of systems to yield a variety of products for application in any of several beneficial processes as described herein. Sufficiently dried solids can be gasified as described herein in more detail. Higher moisture solids might be gasified in a plasma system or partially dried to produce animal bedding or processed using vermiculture, after pasteurization, to generate high nutrient, pathogen free soil amendment.

Anaerobic digestion of organic wastes can be accomplished in a variety of modalities, either batch or continuous loading including, but not limited to: two-phase acid/gas digestion, fixed film, plug flow, complete mix, induced blanket, or other suitable type of digester. This treatment of the organic constituents of the waste streams inputted to this process can yield multiple benefits without negative environmental impact. As described in more detail herein, methane content can be recycled through system processes with or without an increase in concentration.

An anaerobic digester is an apparatus for the anaerobic digestion of organic slurries and can include an enclosed digester tank (fixed or flexible roof), internal media for biofilm development, a biogas collection and flare system, various pumps, and hydraulic control systems. The media can have substantially vertically-oriented, uninterrupted channels to promote enhanced bacterial attachment and biofilm development. The immobilization of microbial biomass within the reactor as a biofilm allows effective treatment of the wastewater at ambient and higher temperatures, as well as reasonable hydraulic retention times. The composition and concentration of bacterial groups in the biofilm developed on the media in the fixed-film digester design expands the potential application of anaerobic digestion to dilute waste with significant levels of suspended solids. This holistic treatment system not only stabilizes the wastewater but also produces energy (biogas), controls odors, reduces pathogens, minimizes environmental impact from waste emissions, and maximizes fertilizer and water recovery for reuse.

Gases produced through the application of anaerobic digestion can be applied to system processes in a number of ways. In the process of FIG. 1 for example, at least a portion of H₂S can be removed from the biogas produced in step 18 (step 20). Particularly, for example, biogas can include H₂S, CO₂, and methane among other materials. Biogas can be scrubbed for the removal of H₂S, a fundamental component of sulfuric acid having numerous industrial applications, including the production of biodiesel. At step 22, an algal growth process can be applied to the biogas after removal of the H₂S. Biogas processed by the algal growth process can have a significant portion of its CO₂ component removed by microalgae with methane at or near pipeline grade remaining. Thus, the methane concentration is increased. The methane gas can be marketed off-site or fired in a pasteurization of excess process water prior to discharge or the direct production of electricity through the production of steam by firing. The heat energy may also be recovered for other system processes. In one example, the heat energy may be employed for heating organic waste preparatory to the anaerobic digestion process described herein.

At step 24, an algal growth system is applied for sequestering system-produced CO₂. The algal growth system may include, but not be limited to, bioreactors, photobioreactors, lighting fixtures, pumps, CO₂ injection apparatus, and other suitable ancillary equipment. Some or all of the system components may be enclosed in a fixed structure. Carbon dioxide can be removed from biogas to increase methane concentration. Carbon dioxide captured following the firing of system produced methane gas for power generation, water pasteurization or other beneficial use can be sequestered by a microalgae growth system. Nutrient uptake by microalgae can reduce the burden on other system processes for the removal of nutrients and contaminants from process water before pasteurization and discharge as a potable equivalent. Sequestration of carbon dioxide in this manner creates hydrocarbons in the form of algal lipids and cellulosic materials that are suitable for gasification following harvesting and drying. These processes provide the benefit of internal recycling for use in internal system processes. In recognition of the fact that continuous internal recycling may yield quantities of microalgae in excess of that which is necessary to generate system products, harvested algae from such an enclosed system can be alternatively processed to produce biodiesel and ethanol with co-products useful in pharmaceutical/nutraceutical applications.

While it is recognized that enclosure of an algal growth system in a fixed structure may require significant energy expenditure to produce the light required for photosynthesis, this expense is offset by the recognition that the growth day can be uninterrupted by circadian rhythms or adverse weather conditions. Such a facility can also protect bioreactors or photobioreactors from degradation due to exposure to environmental elements.

Fixed structures comprising the facility in its entirety can apply two waste elimination strategies. All fixed structures can have concentrated solar power and/or photovoltaic systems installed on all rooftops as well as drain systems for rainwater reaching the rooftops that divert rainwater to the system intake for recapture as a resource.

As is evident to those of skill in the arts applied in this process heat, in the form of exhaust from combustion engines/turbines, gasification, pasteurization of excess process water, FTS treatment of syngas and other sources is widely available throughout this process. In keeping with the fundamental tenet that waste is a resource out of place, this heat is by design, not waste but a resource in the production of electricity through the operation of a Rankine Cycle system, such as an Organic Rankine Cycle (ORC) system. This residual heat can also be used to elevate the temperature of incoming organic wastes to facilitate anaerobic digestion, maintain optimal temperatures for algal growth through maintenance of water temperature and reduced heat loss from bioreactors or photobioreactors through the temperature control of the facility in which they are housed.

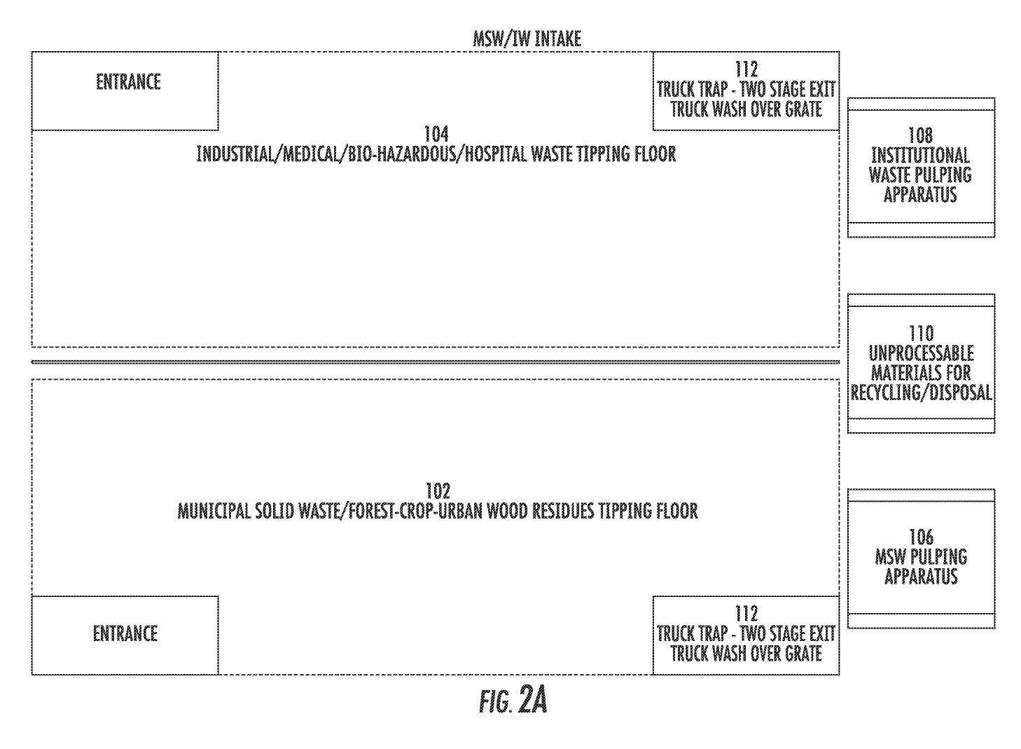

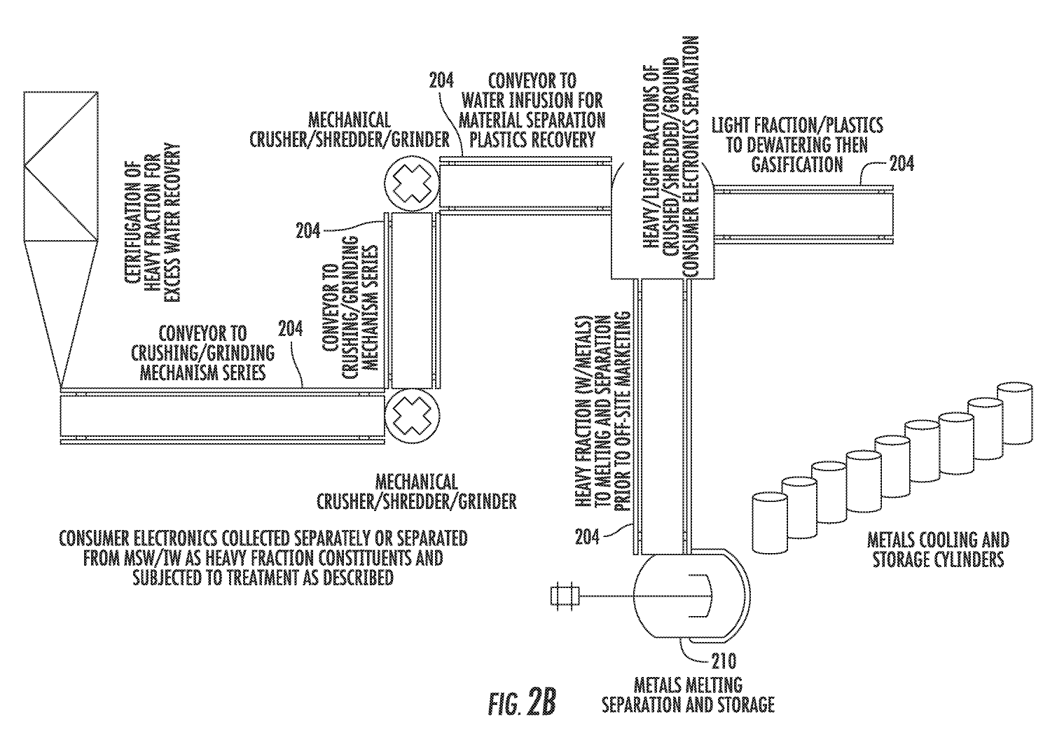

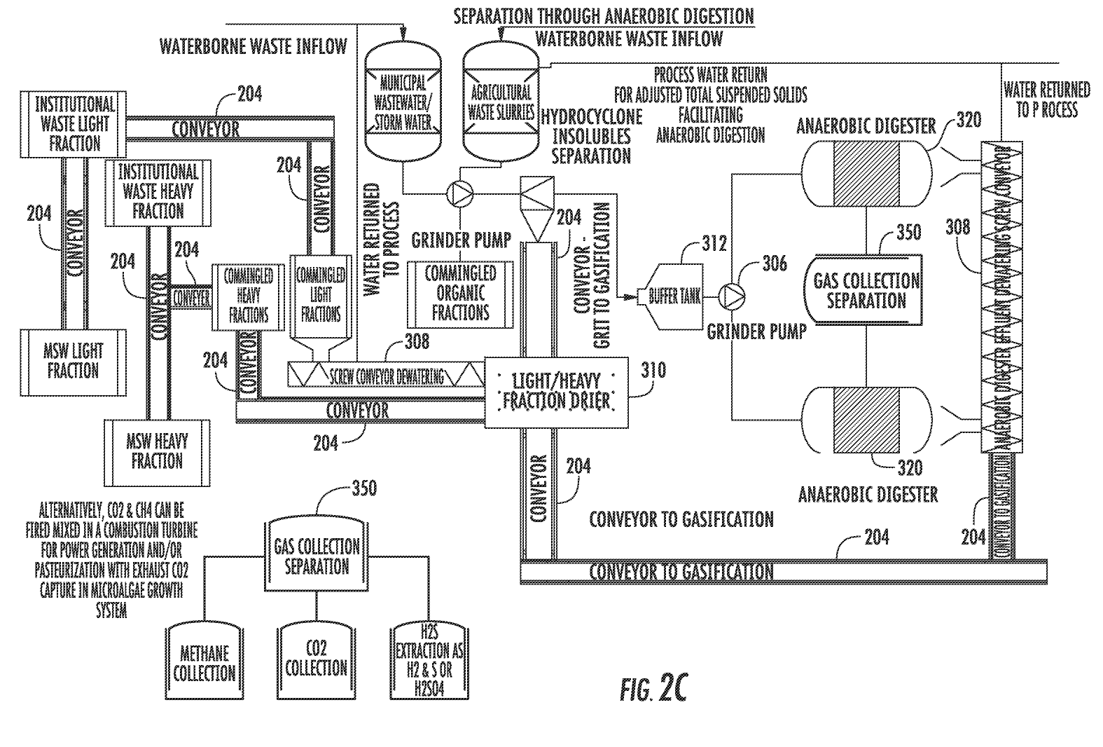

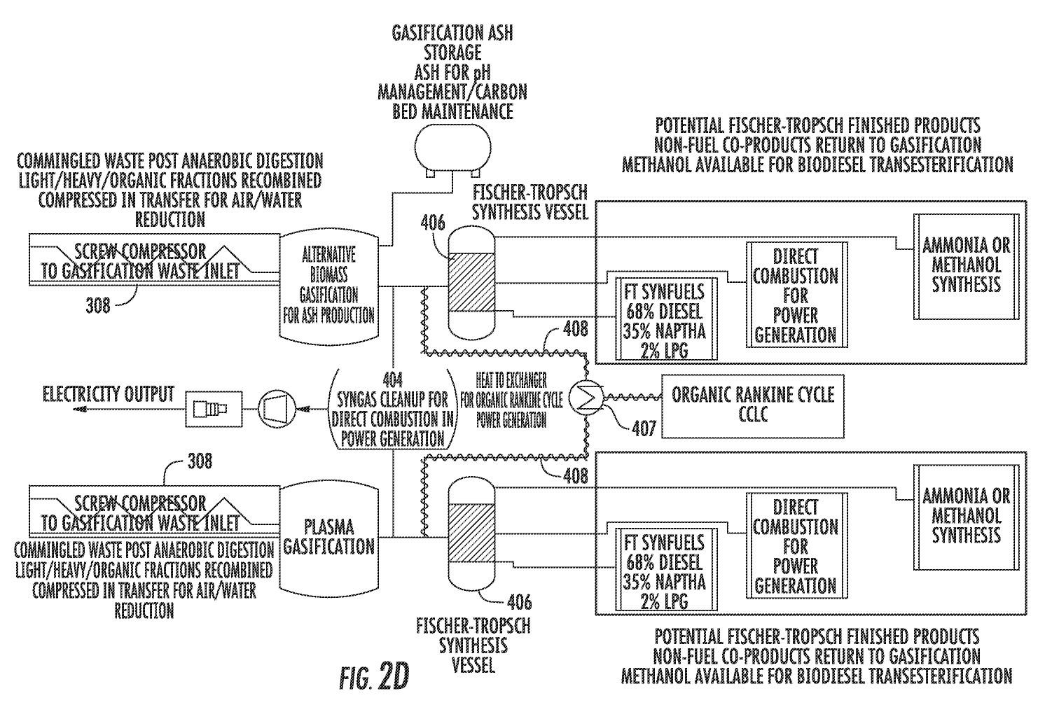

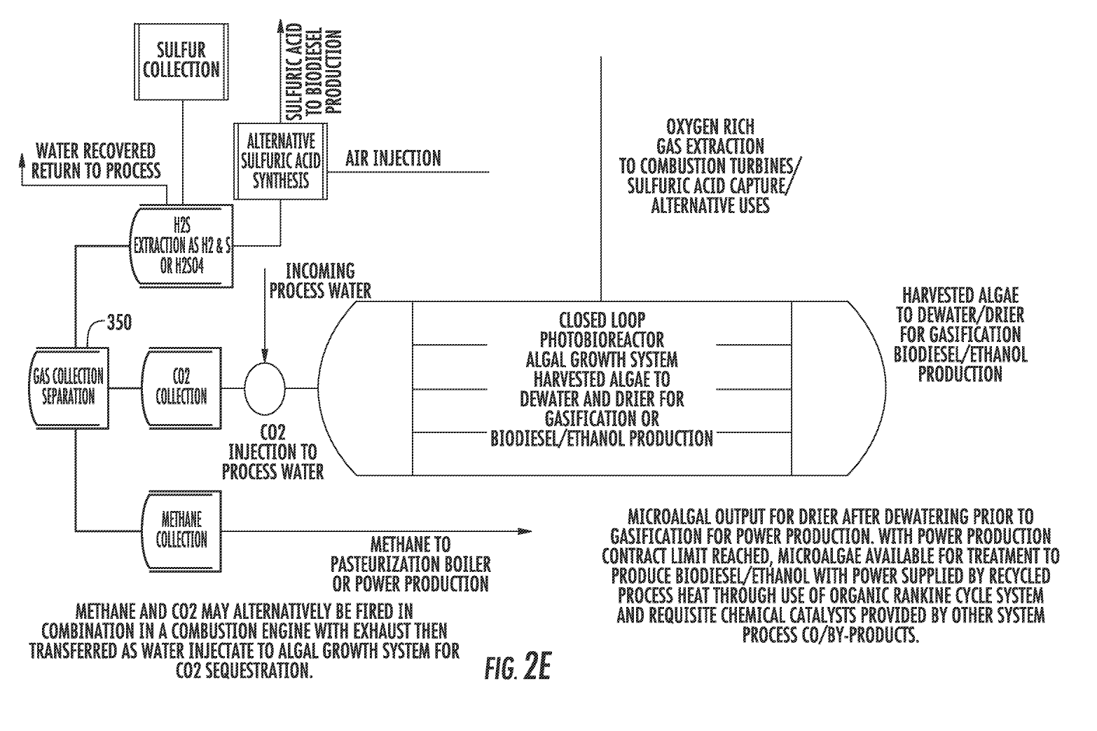

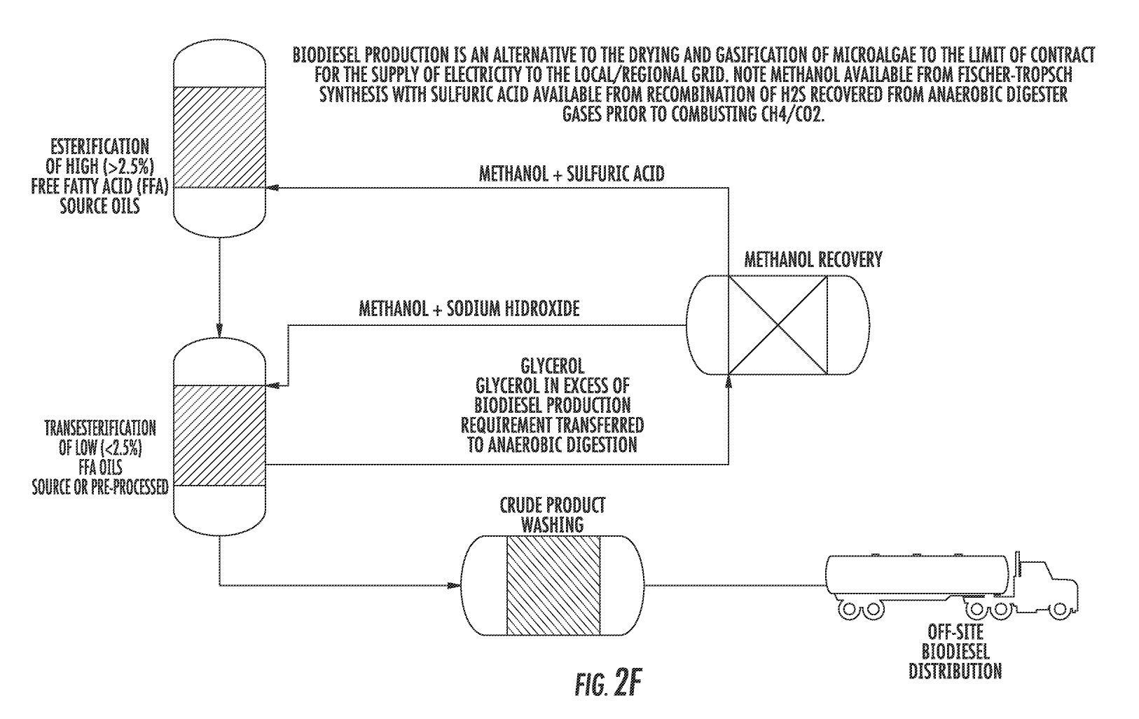

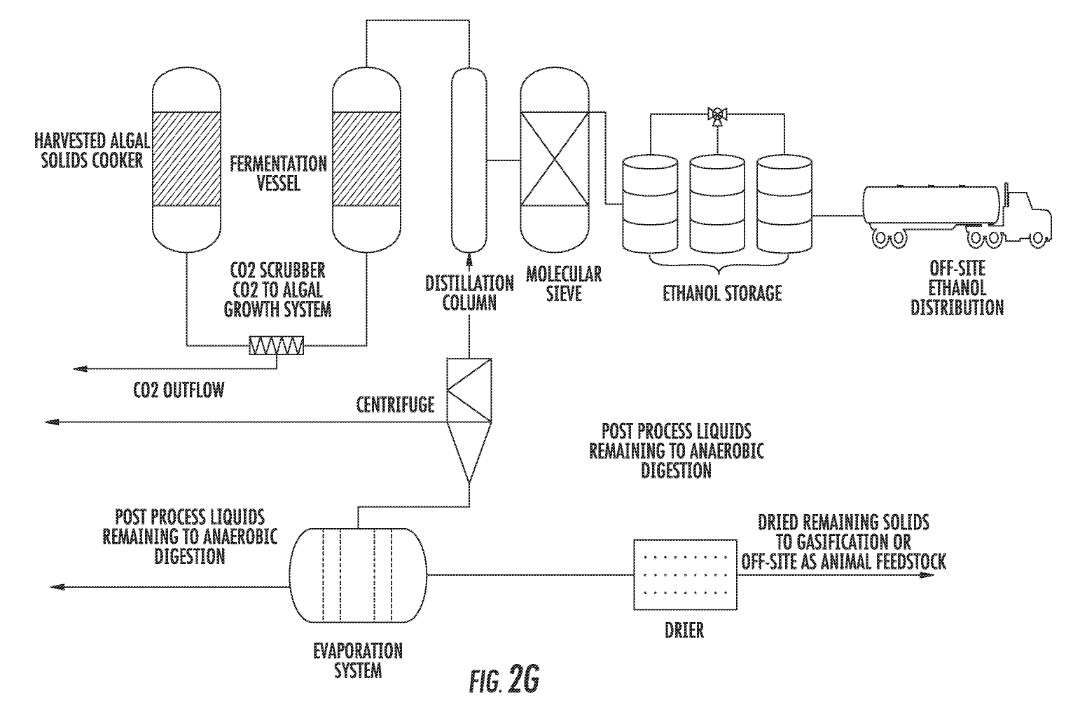

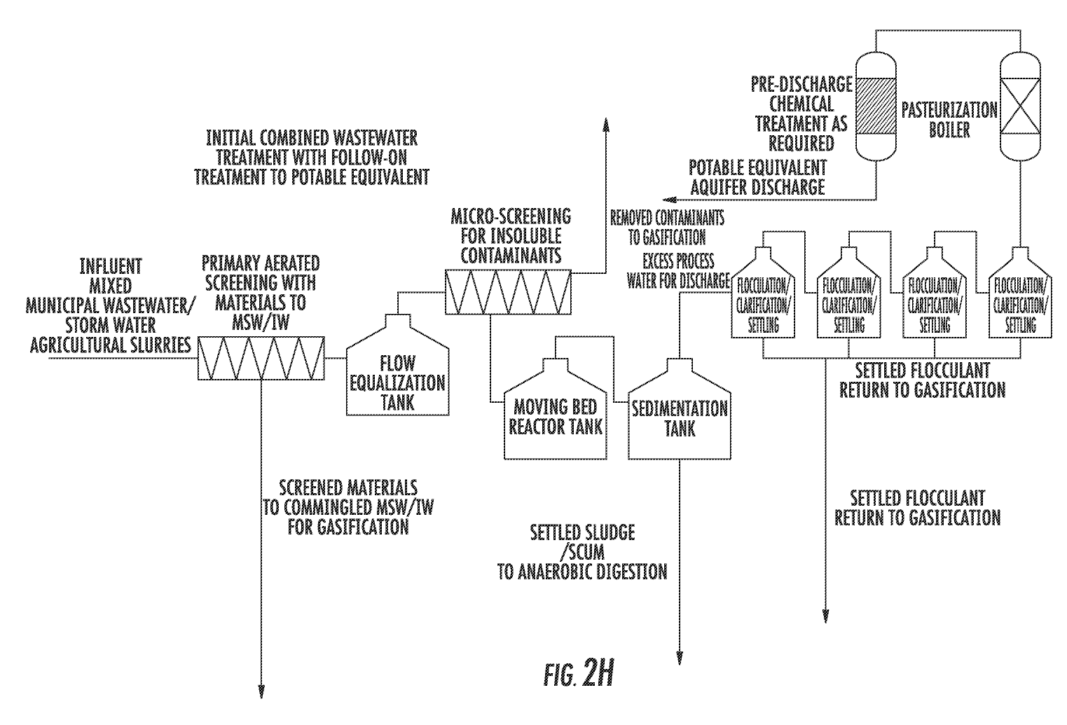

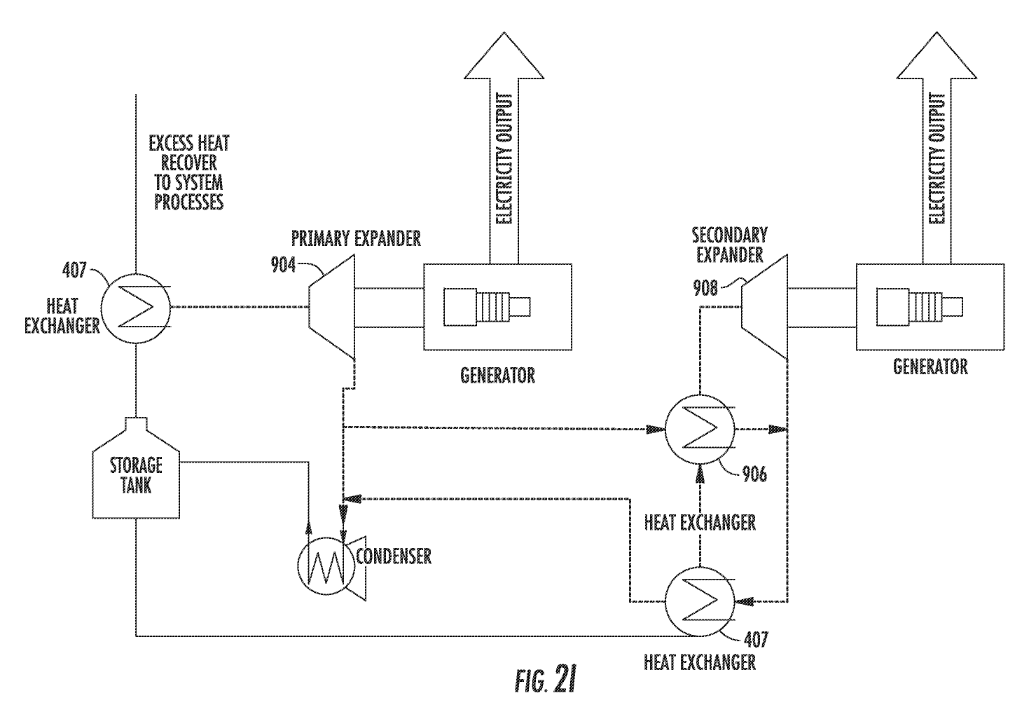

FIGS. 2A-2I illustrate a block diagram of an exemplary system for generating resources using wastes according to an embodiment of the subject matter disclosed herein. The exemplary system can function as described herein below with the understanding that each facility may be configured differently, even to the point of exclusion of a process or processes deemed unnecessary in a given application, to incorporate evidence based best practices suited to its individual waste stream. Referring particularly to FIG. 2A, intake or receipt of wastes can be performed in a manner consistent with the processes known to those of skill in the art. Waste collection trucks can be unloaded inside a divided building, in a tipping floor area 102. Industrial waste and hospital/bio-hazardous/medical wastes (hereinafter referred to collectively as industrial wastes) can be unloaded on the opposite side of a divided building in another tipping floor area 104. In either side of the building, the access doors can be closed once a collection truck is inside. The division of the building into two sides eliminates the risk of contaminants from industrial/medical/hospital/bio-hazard waste producers from contacting collection vehicles operating in residential areas. The operator of the truck can be directed to a designated location by a tipping floor manager where wastes can be off-loaded. A tipping floor manager can assist a truck operator in off-loading, verify the acceptability of the waste and prevent or reduce the risk of incoming waste contacting the vehicle. Following off-loading, a truck can pass through an automated sprinkler system as it proceeds in a forward direction to wash away any waste that may have inadvertently contacted the vehicle.

Tipping room personnel can be trained to identify and deal with unacceptable materials. When unacceptable materials, such as tanks for the storage of pressurized gases, are identified on the tipping room floor, the material can be segregated from other incoming wastes and transferred to an appropriate reuse, recycling or disposal facility 110.

The incoming wastes can then be prepared for processing. The first stage of the processing of wastes can be designed using an evidence-based waste profile to determine the normal characterization of the waste stream as the underlying data upon which the development of the front end system to free the waste for processing can be designed. Industrial wastes can be processed using processes mirroring those for the processing of wastes in the area designed to accept these wastes.

Waste pulping can be accomplished using techniques well known to those of skill in the art. For example, waste pulping can occur at a waste pulping apparatus 106 and/or an institutional waste pulping apparatus 108. A waste pulping component can be used to process comingled MSW and industrial wastes in an alternative to segregated pulping treatment. A company having experience in waste pulping is BTA International. Industrial wastes can be processed in a like manner to extract biodegradable materials. Pulping of incoming wastes can function to separate the waste into three distinct fractions. A light fraction can include plastics, textiles and small wood particles, an organic suspension, and a heavy fraction composed of metals, glass, stones, batteries and other materials. The pulping equipment can use hydraulic shear to break down the fibrous organic waste into a pulp. Shredding the waste can compromise the structural integrity of the organic fibers releasing toxins that might be present in discarded items such as batteries. Using this process in place of shredding can avoid this potential environmental hazard.

The components shown in FIG. 2B can be used for processing collected consumer electronics and other heavy fraction materials. Referring to FIG. 2A, the heavy fraction can be collected in a trap located at the bottom of an inclined floor at the bottom of one or both of pulpers 106 and 108. The pulpers 106 and 108 can be equipped with scrapers operable to rotate with an agitator and continuously scrape settled heavy material toward the trap. The trap can be configured with upper and lower gates. An upper gate can open to permit the passing of heavy materials into the trap, and a lower gate can remain closed to secure the waste in the trap. Process water can be used to flush the materials collected in a trap to prevent the build-up of organic materials within the trap and to clean the materials collected. Cleaned heavy fraction materials can be withdrawn by closing the upper gate and opening the lower gate after which such material can be transferred to a collector for separation of recyclable ferrous and non-ferrous metals with the remaining waste returned to the stream in preparation for treatment by gasification later in the process. After separation, heavy fraction constituents can be crushed and ground to release plastics and other lighter materials through the infusion of water to facilitate separation at which point the lighter materials can be comingled with other light fraction constituents and remaining heavy materials further processed for recovery of recyclable metals prior to gasification. It is noted that these constituents may already have been separated from high plastic content heavy fraction materials.

A light fraction, including primarily plastics, can be separated from the organic fraction by the use of a mechanically operated rake to capture floating debris in pulpers 106 and 108. Materials so collected can be transferred to a screw conveyor or press to dewater the material prior to drying and shredding preparatory to gasification as described herein.

Grit, in the form of sand, cement or other fragments can be removed by a hydrocyclone system 304. The hydrocyclone system 304 can use centrifugal forces to separate grit, comprised in part of sand, tile, grout, from the organic suspension. The bottom of the hydrocyclone system 304 can be angled to permit dropping of these materials into a screening mechanism or classifying pipe wherein clean backwash water can be infused to flush residual organics from the grit box. The grit box is can be discharged whenever grit content reaches a predetermined level. The grit can be collected in a screw conveyor and transferred into a container for recycling or disposal.

Following separation of all contaminants, the organic suspension can be pumped into a pulp buffer tank 312. The pulp buffer tank can enhance consistency of hydraulic loading and facilitate the creation of a homogeneous organic slurry.

An organic fraction can be transferred from the buffer tank 312 and subjected to anaerobic digestion at anaerobic digesters 320 for treatment. Anaerobic digestion can be a process in which biodegradable organic materials provide feedstock for a consortium of bacteria in the absence of oxygen, can be accomplished using any of a variety of digester technologies, such as plug-flow, complete-mix, covered lagoons or any of several demonstrations of mixed reactors with flexible-film support media, all of which are variations of batch and semi-continuous processes and well known by those of skill in the art; however, the function of such a process should not be limited to this description as understood to those of skill in the art. In an exemplary embodiment of the presently disclosed subject matter, fixed film anaerobic digestion can prove to best process wastes thus far prepared. A fixed film digester of the type developed and employed by the University of Florida's Research Foundation can function to process organic wastes yielding biogas with an effluent remaining that has multiple potential applications downstream of the digestion process. The effluent can be dewatered using centrifugation or screw presses 308 or other suitable dewatering or drying technology. This process can significantly reduce the moisture content of the extracted solids while reducing solid contamination of process water to less than five percent by weight.

Gases, such as biogas, produced through the anaerobic digestion process are composed of approximately 55-65% methane (CH₄), 35-45% carbon dioxide (CO₂) and trace gases including hydrogen sulfide (H₂S). At a gas collection separation system 350, gases produced in the anaerobic digestion process can be separated into their component gases, specifically CH₄ and CO₂, collected and used in other system processes. Methane can be fired to produce electricity and heat for water pasteurization prior to discharge as potable equivalent to a regional aquifer. CO₂ can be used to nourish an enclosed microalgae growth system. CO₂ produced through the firing of methane can be added to that produced by the anaerobic digesters 320 to further enhance microalgal growth. H₂S can be collected and chemically treated to produce water and sulfur using methods known to those of skill in the art or, in an alternative, to yield sulfuric acid, a valuable industrial chemical with uses that include the production of biodiesel. In an alternative, these gases can be collected, scrubbed for the removal of H₂S as later described and delivered to an enclosed microalgae growth system for uptake of CO₂ by microalgae yielding a near pipeline grade methane gas.

Dewatered anaerobic digester effluent can be subjected to drying by methods known widely to those of skill in the art to have moisture content reduced to less than 20% to facilitate its introduction to a gasification process. This dried material, infused and compressed into other wastes prior to gasification can displace entrained air leading to an increase in the thermal efficiency of a gasification process so employed.

Biogas has a high heating value (HHV) and is possessed of very low contaminant levels. After the removal of water and H₂S, the gas requires no further processing prior to combustion in co-generation engines. Exhaust gases from this implementation, predominantly CO₂, can be captured and infused into process water to transit a microalgae growth system for the purpose of nourishing the algae therein. Such water can be repeatedly recycled through this process, having CO₂ infused at the intake of the algal growth system with reduced CO₂ water outflow re-heated and recycled. Such a configuration can maintain high relative CO₂ levels and adequate temperature in this medium yielding more rapid algal growth rates than currently experienced in systems employing lower CO₂ levels in the treatment of flue gases. Heat produced by a combustion power generation system can be used in the production of additional power through the implementation of a Heat Recovery Steam Generator (HSRG) system, such as a Rankine Cycle System 900. This heat also has potential application in the maintenance of water temperature in an algal growth system, temperature elevation of influent to an anaerobic digestion process and other system heat requirements.

Excess water recovered from the solids/liquid separation of the digestate, biogas dewatering and dewatering of light and heavy fractions and can be stored in a buffer tank. This water can be recycled as process water throughout the process herein described to reduce the commitment of external water resources to system use. Water in excess of process requirements can be discharged as potable equivalent to the regional aquifer following secondary and tertiary treatment by processes, including pasteurization, well known to those of skill in the art to yield water of this quality.

Digested solids remaining after dewatering can be further dried to facilitate gasification as later described, subjected to vermiculture to fix nutrients remaining to produce a slow-release soil amendment that can reduce run-off from agricultural operations, be transferred off-site as animal bedding or used to make a final compost product. Following anaerobic digestion, approximately 85% of the biological activity is expected to be completed. In a composting option, aerobic finishing of the digestate product would be necessary and would require the addition of bulking materials to produce greater porosity and oxygenation during the aerobic finishing of the digestate. Alternatives to composting reduce the hazards associated with aerobic digestive processes and include gasification or the application of vermiculture or other processes as elsewhere described herein.

Gases and air from process vessels and fixed structures not otherwise applied to system processes can be collected and treated in a biofilter prior to release to the atmosphere. This treated air can meet or exceed air quality regulatory requirements. In normal operation, biogas produced can be combusted in cogeneration units which comply with, or exceed emissions requirements or used to generate heat required for pasteurization or other system process. In the event of an emergent shutdown of power production units, high carbon biogas can be inputted to a gasification process for the production of a synthesis, or producer gas (syngas). Alternatively, to prevent the build-up of hazardous gases under pressure, the biogas can be combusted in a fully enclosed flare. Through the operational design of conversion of waste to a liquid pulp, odors produced through biological activity are fully controlled and the occurrence of airborne particulate in the form of dust is significantly reduced.

An algal growth system for the propagation of microalgae in a controlled environment within a fixed structure can be used to sequester CO₂ while producing valuable product for system re-use or available to off-site markets. Bioreactors or photobioreactors are fermenters in which phototrophic microorganisms, such as algae, microalgae, cyanobacteria and purple bacteria are cultivated, that is to say in which both the growth and the propagation of these cells is made possible or the production of various substances is promoted by means of phototrophic cells. The microalgae include, on the one hand, the prokaryotic cyanobacteria as well as eukaryotic microscopic algae classes. These organisms supply a wide variety of substance classes that can be used for production of biodiesel, ethanol, pharmaceutical, cosmetic, nutritional and animal nutrition purposes and for technical purposes (for example, heavy-metal adsorption). Important substance classes in this connection are lipophilic compounds, such as, for example, fatty acids, lipids, sterols and carotenoids, hydrophilic substances such as polysaccharides, proteins or amino acids and phycobilin proteins (pigments), and also the total biomass as protein-rich raw material low in nucleic acid.

The growth of microalgae in bioreactors or photobioreactors is well known to those of skill in the art. CO₂ for nourishment of a selected algal species can be sourced from the output of other apparatuses as elsewhere herein described. Thermal balance can be obtained through the operation of heat exchangers to maintain an optimal temperature both within a bioreactor or photobioreactor system and its environs to facilitate growth of a selected algal species.

In the practice of the presently disclosed subject matter, algal lipids in the form of oils extracted from algae following harvest from an enclosed bioreactor or photobioreactor is the main raw material used to prepare the present green biodiesel. In transesterification of algal oil the alcohol of choice is normally ethanol or methanol. Methanol can be a co-product of the Fischer-Tropsch synthesis process and so would, in the presently disclosed subject matter, be a readily available resource without having to transport a potentially hazardous chemical. When a choice of vegetable oil is necessary to replace or supplement algal oil, the most preferred oil source in the U.S. is soybean oil, whereas rapeseed oil is the preferred oil in Europe, and palm oil and castor oil are prevalent in Asia.

Biodiesel is a mixture of fatty acid alkyl esters produced, ordinarily, from a plant source. Typically a mixture of methyl or ethyl esters is produced from a transesterification reaction involving triglyceride esters, vegetable oil and an alcohol, ordinarily methanol or ethanol, which yield glycerol as a by-product. When the reaction involves free fatty acids specifically, it is an esterification rather than a transesterification, as those skilled in the art understand. The methyl and ethyl esters are similar to petroleum diesel in structure and properties as fuels, which makes biodiesel suitable for routine use in present-day diesel engines.

The transesterification reactions of fatty acid-containing raw materials and one or more alcohols take place in either batch or continuous reaction equipment as is used for biodiesel production known to those of skill in the art. The fatty-acid starting materials can be literally any fatty-acid rich material such as algal oils, vegetable oil, used vegetable oil, restaurant waste grease, or surplus liquid or solid fats such as vegetable shortenings, surplus margarine or similar fatty acid compositions. Any animal or vegetable fat or oil may be used, with additional processing if necessary to accommodate its characteristics according to the skill of the art. For example, restaurant waste grease requires simple extra processing before reaction to remove excess water and to filter out precipitates and other sludge, but to do this is already known in the biodiesel arts. The alcohol reactant can be literally any alcohol, although methanol, ethanol, propanol and butanol are preferred and methanol and ethanol are most preferred. A mixture of two or more alcohols may be used and, in certain instances, C₁-C₄ diols may be used although mono-ols are preferred. Volume of reaction is unlimited as long as effective stirring of the reactants can be achieved, either by a mechanical stirrer in a batch reactor or by appropriate drumming or tumbling in a continuous reactor. In lieu of stirring per se, it is possible to use a continuous packed-bed reactor known in the art, which obviates either stirring or tumbling. Proportions of alcohol to fatty acid raw material are preferably about 40:1 but can range within 35:2 and even 30:3 or any ratio in between or that will yield a suitable transesterification. The amount of solid catalyst to use can vary depending on the reactants. In many cases, as a practical matter, the solid catalyst comprises about 20% by weight of the reaction mixture. Choice of quantity of solid catalyst is known in the art, and this operational component of the presently disclosed subject matter inheres in the choice of the actual catalyst to improve the overall production of the present biodiesel. Although other physical forms of solid catalyst may be used, typically the solid catalysts of the operational component of the presently disclosed subject matter are provided in a particle or powdered form with particle sizes and particle size distributions typical of solid catalysts in industry. The governing parameter for particle size and particle size distribution is not any particular range, the design of which is well within the ordinary skill of the art. When continuous reactors are used the catalyst(s) are generally immobilized in a matrix bed and provided in line for plug flow. Immobilization of solid catalysts on a matrix for plug flow is well known in the industrial arts. Reaction temperatures are generally between about 60°-450° C., preferably 70°-300° C., and most preferably 150°-260° C. Of note is the availability of process heat for the production of biodiesel using such technology as described herein reduces the energy import required by most such production facilities. The reaction may be conducted at ambient or elevated pressure (anywhere between 1 and 500 atmospheres, more preferably between 1 and 70 atmospheres) and with or without typical biodiesel manufacturing co-solvents such as hexane. Reaction times range from 5 minutes to one hour and can even be 5-20 minutes and in many cases 5-10 minutes.

An exemplary embodiment of the presently disclosed subject matter includes the operation of a process for forming ethanol from algae, comprising: (a) growing starch-accumulating, filament-forming or colony-forming algae in an aqua culture environment; (b) harvesting the grown algae to form a biomass; (c) initiating decay of the biomass; (d) contacting the decaying biomass with a yeast capable of fermenting it to form a fermentation solution; and, (e) separating the resulting ethanol from the fermentation solution.

Examples of the starch-accumulating filament-forming or colony-forming algae include an algae in a phylum selected from Zygnemataceae, Cladophoraceae, Oedogoniales, Ulvophyceae, Charophyceae, or a combination thereof. Examples of the starch-accumulating filament-forming or colony-forming algae include an algae selected from spirogyra, cladophora, oedogonium, or a combination thereof.

Initiating decay, as used herein, means that the biomass is treated in such a way that the cellular structure of the biomass begins to decay (e.g., cell wall rupture) and release the carbohydrates contained therein. Initiating decay can be accomplished mechanically, non-mechanically, or a combination thereof. Mechanical initiation means that the biomass is subjected to some sort of mechanical distortion that begins the decay process. Examples of mechanical initiation can include stirring, chopping, crushing, blending, grinding, extruding, and shredding. The mechanical initiation can be performed (a) under ambient light and atmosphere, (b) in the dark under an ambient atmosphere, and (c) in the dark and under anaerobic conditions (see below). Non-mechanical initiation means that biomass is placed in an environment where the presence of light and oxygen is limited or entirely absent so as to initiate and promote the decay of the biomass (e.g., a dark and anaerobic environment). The amount of light present will typically be so low that photosynthesis cannot continue. For example, the biomass can be placed in water under cover (e.g., inside a tank) where sunlight is prevented from contacting the biomass. Also, a gas (e.g., carbon dioxide or nitrogen) can be bubbled through the biomass and water to reduce or remove the presence of air. The cellular structure of the biomass under dark and anaerobic conditions is expected to begin to decay (e.g., cell wall rupture) and release the carbohydrates contained therein. This decay can occur without mechanical assistance, though mechanical assistance as described above can also be used. As an example, the biomass can be maintained under decay conditions for as little as one or as many as seven or more days. The vessel, structure, etc. housing the biomass under decay conditions can be fitted with a sensor (or sensors) that will allow for sampling of the biomass to determine its level of decay. The sampling can be done by methods known to those of skill in the art to show when the cell walls of the algae have ruptured or are rupturing. For example, a sample of the biomass can be observed under a microscope to determine if the desired cell wall rupturing has been achieved. It may be desirable to wait until cell wall rupturing is observed before contacting the biomass with the fermentation yeast.

One of ordinary skill in the art would recognize that the quantity of yeast to be contacted with the biomass will depend on the quantity of the biomass present as well as the rate of fermentation desired. The yeasts used are typically brewers' yeasts. Examples of yeast capable of fermenting the decaying biomass include, but are not limited to, Saccharomyces cerevisiae and Saccharomyces uvarum. Besides yeast, genetically altered bacteria known to those of skill in the art to be useful for fermentation can also be used. The fermenting of the biomass can be conducted under standard fermenting conditions.

Separation of the ethanol from the fermentation solution means that once ethanol begins to form from fermentation, it is then isolated from the fermentation solution. It is expected that the fermentation solution would contain at least water, ethanol, and the remaining biomass. Separation can be achieved by any known method (e.g. distillation). The separation operation can be performed on the liquid and solid portions of the fermentation solution (e.g., distilling the solid and liquid portions), or the separation can be performed solely on the liquid portion of the fermentation solution (e.g., the solid portion is removed prior to distillation). In addition, separation of ethanol can be performed on an entire batch of fermentation solution. Alternatively, separating can entail removing the fermentation solution portion-wise (e.g., 1, 2, 3, 4, 5, 6, 7, 8, 9, 10, 20, 30, 40, 50% or more of the solution at a time) or continuously and replacing it with additional fermentation solution or liquid (e.g., water). The separated ethanol, which would not generally not be fuel-grade, can be concentrated to fuel grade (e.g., at least 95% ethanol by volume) via methods known to those of skill in the art (e.g., a second distillation).

One of ordinary skill in the art recognizes that the level of ethanol present in the fermentation solution can negatively affect the yeast/bacteria. For example, if 17% by volume or more ethanol is present, then it will likely begin causing the yeast/bacteria to die. It can therefore be desirable to begin separating ethanol from the fermentation solution before or at least when ethanol levels less than but not equal to or greater than 17% by volume (ethanol to water) are observed. Ethanol levels can be determined using methods known to those of ordinary skill in the art.

The fermentation reaction can be run 1, 2, 3, or more times on the biomass. For example, once the level of ethanol in the initial (i.e., first) fermentation solution reaches 12-17% by volume, the entire liquid portion of the fermentation solution can be separated from the biomass or the fermentation solution in total (i.e., biomass and liquid portion) can be run through a procedure to isolate the ethanol (e.g., distillation). The once-fermented biomass can then be contacted with water and yeast/bacteria to form a second, third, etc. fermentation solution. This process can be repeated until the yield of ethanol is undesirably low. Typically, after the first fermentation run, the amount of biomass remaining in the vessel holding the biomass (e.g., tank) is determined. If less than half of the starting biomass remains, then it is assayed for carbohydrate level. If sufficient carbohydrates remain, then additional biomass is added and the fermentation process repeated. If sufficient carbohydrates do not remain, then the biomass is removed (e.g., pumped) from the vessel for further processing as anaerobic digester feedstock or subjected to drying technology preparatory to implementation as gasification feedstock.

It may be desirable to isolate or harvest the yeast/bacteria from the fermentation reaction for recycling. The method of harvesting will depend upon the type of yeast/bacteria. If the yeast/bacteria are top-fermenting, then it can be skimmed off the fermentation solution. If they are bottom-fermenting, then it can be removed from the bottom of the tank.

A by-product of fermentation is carbon dioxide. During the fermentation process, it is expected that about one-half of the decomposed starch will be discharged as carbon dioxide. This carbon dioxide can be collected by methods known to those of skill in the art (e.g., a floating roof type gas holder). The collected gas can then be beneficially supplied to the algae aqua culture as an inorganic carbon source for growing the algae.

Lipids/oils, which are useful for forming biodiesel typically remain in the biomass after it has been subjected to fermentation and the fermentation solution has been removed. These lipids/oils can be isolated from the biomass and then used to form biodiesel using methods known to those of skill in the art to form biodiesel. A convenient method of separating lipids/oils from the biomass is by pressure. For example, the biomass can be pressed and the resulting lipid-rich liquid separated.

The term “reactor” as used herein refers to the process containment vessel, or furnace, into which refuse, e.g. municipal solid waste, is placed and heat is added for the purpose of promoting the simultaneous pyrolysis of organics and vitrification of inorganics of the mixed wastes.

It is widely recognized that if waste is transported to a central location, pyrolysis and vitrification can be accomplished, utilizing plasma arc heating technology, in an efficient and safe manner and useful gaseous and vitrified products produced so as to avoid placing the waste residue into a landfill. The presently disclosed subject matter presents a versatile method for the handling of mixed waste which improves on earlier systems and which can be adapted to the requirements of the particular quantity and mixed characteristics of the wastes to be processed.

Plasma arc heated processes are receiving considerable attention for waste treatment over fuel combustion heated processes because of several distinct advantages of plasma heat, which is well suited for the pyrolysis and vitrification of waste materials. A plasma arc torch operates by supporting a high voltage electric arc on a flow of plasma (ionized) gas to generate an extremely hot “flame”. The quantity of plasma gas flowing through the plasma torch is significantly less than the quantity of gas required to release the equivalent heat energy by the combustion of hydrocarbon fuels. A further difference and advantage of a plasma torch heat source over a combustion heat source is that the plasma torch can be used to produce useful by-product gases of higher caloric content referred to here as the degassing process. In addition, by virtue of the fact that a plasma arc torch uses only a small quantity of gas to support the arc and generate the heat, combustion is unlikely to occur spontaneously in the materials which are being heated. A major advantage of the plasma torch is that it is capable of unusually high rates of heat transfer, adding to its inherent efficiency. In addition, the temperature of 4,000°-7,000° C. generated by a plasma torch is much hotter than that generated by a combustion source and is hot enough to melt any known material simultaneously with the pyrolysis degassing process.

Plasma gasification processes, well known to those of skill in the art can be employed in the conversion of wastes to useful products. The Solena Group, Plasco Energy Group and Westinghouse, among others are corporate entities involved in the employment of plasma gasification technology for the treatment of mixed wastes and production of a synthesis gas comprised of hydrogen (H₂) and carbon monoxide (CO) useful in the production of electricity or further treatment through Fischer-Tropsch synthesis.

The presently disclosed subject matter also relates to the conversion of mixed wastes to useful products. Addressing this fundamental goal, a component of the presently disclosed subject matter relates generally to the field of Fischer-Tropsch synthesis. More specifically, a component of the presently disclosed subject matter relates to a system and method for converting syngas to liquid hydrocarbons via Fischer-Tropsch synthesis.

First developed in Germany in the third decade of the twentieth century, FT synthesis has long been used to convert carbonaceous wastes to valuable products.

The process of converting the available material to a liquid fuel involves the partial oxidation of the material prior to a catalyzed reaction to create a liquid fuel. Referring to FIG. 2D, FT reactors 406 can provide catalyzed synthesis of synthetic petroleum substitute liquid fuels. The process occurs via a catalyzed chemical reaction in which carbon monoxide and hydrogen from the material are converted into liquid hydrocarbons. The reaction is highly exothermic, and requires a cooled reactor to maintain conditions favorable for continued synthesis. Commercial FT reactors 406 require cooling liquid to be transported through heat exchanging conduits 408 in order to remove heat from the reactor. This cooling requirement is of benefit to the presently disclosed subject matter in that recovery of the energy from process heat through the implementation of Rankine Cycle system 900 can yield further benefit for the presently disclosed subject matter.

Additionally, a desirable liquid or wax co-product is created during the FT reaction that must be separated from the catalyst, intermediate hydrocarbon, and associated gases in the reaction slurry, which may be returned to the reactor. Current reactor systems use a number of processes in this separation step including filtering, and settling.

As is apparent to those of skill in the arts applied in the process disclosed herein, heat is produced in a number of the apparatuses applied in this process in excess of that necessary for the operation of each apparatus. This heat, generally regarded as waste heat in most settings can be captured and applied throughout this process for multiple uses. Through the implementation of heat exchangers throughout the process disclosed herein all heat energy not required for operation of the process or apparatus in which it is generated can be recovered for use thereby providing a necessary heat sink while increasing the thermal efficiency of all components of the process disclosed herein. These applications include, but are not limited to; power production, temperature maintenance in critical functions such as; elevation of influent temperature prior to anaerobic digestion to facilitate the digestive process; maintenance of appropriate water temperatures for an algal growth system; maintenance of ambient temperature of a fixed structure housing an algal growth system to reduce heat loss through radiation to its environs from such a system; pasteurization of potable equivalent effluent prior to discharge.

Rankine cycle power plants operating with an organic working fluid are known in the art. Such a power plant comprises a boiler for vaporizing the working fluid, a turbine responsive to vaporized working fluid produced by the boiler for expanding the vapor and producing work, a generator coupled to the turbine for converting the work produced thereby into electrical energy, and a condenser for condensing expanded vaporized working fluid exhausted from the turbine and producing condensate that is returned to boiler either by pump or under the influence of gravity. A power plant of this type, hereinafter referred to as an Organic Rankine Cycle (ORC) is a power plant of the type described, is widely commercially available.

A Rankine cycle power plant generally utilizes as a working fluid compounds selected from the group consisting of bicyclic hydrocarbons, substituted bicyclic aromatic hydrocarbons, heterocyclic bicyclic aromatic hydrocarbons, substituted heterocyclic bicyclic aromatic hydrocarbons, bicyclic compounds where one ring is aromatic and the other condensed ring is non-aromatic, and their mixtures.

In an exemplary embodiment of the presently disclosed subject matter, an ORC operated in a manner consistent with that developed and taught by Stinger and Mian efficiently converting waste heat into usable power using a Cascading Closed Loop Cycle (CCLC) hermitically sealed process. The CCLC uses a primary fluid stream such as propane, which is vaporized in a primary indirect heat exchanger 902, expanded in a primary expansion turbine 904, and discharged to a secondary indirect heat exchanger 906 where it is introduced to a stream mixer. The secondary indirect heat exchanger 906 superheats a secondary stream of propane by using the vaporized propane exiting the primary expansion turbine 904. The secondary stream of propane is directed to a secondary expansion turbine 908 for generating useful power. The secondary stream of propane, exiting the secondary expansion turbine 908, is combined with the primary stream of propane in the stream mixer. After mixing in the stream mixer the combined propane stream is directed to a tertiary indirect heat exchanger where heat in the propane stream is transferred to the secondary propane stream in the tertiary heat exchanger. After exiting the tertiary indirect heat exchanger, the combined stream is directed to a condenser where the propane stream is condensed to a liquid and directed to a high pressure pump. The liquid propane stream, discharged from the high pressure pump, is directed to a stream separator where it is separated into the primary propane stream and the secondary propane stream where the cascading expansion turbine closed loop hermetically sealed cycle repeats the vaporization, expansion, liquefaction and pressurization process. The discharge temperature of the waste heat effluent from the primary heat exchanger 906 can be directed to atmosphere through the exhaust stack though its preferred use is as a heat transfer medium for implementation in other system processes.

The primary expansion turbine 904 and secondary expansion turbine 908 can be connected in series or parallel to a power generation device using any speed changing means to produce mechanical or electrical power.

While the embodiments have been described in connection with the preferred embodiments of the various figures, it is to be understood that other similar embodiments may be used or modifications and additions may be made to the described embodiment for performing the same function without deviating therefrom. Therefore, the disclosed embodiments should not be limited to any single embodiment, but rather should be construed in breadth and scope in accordance with the appended claims.

What is claimed:

1. A method for generating resources using wastes, the method comprising:
receiving wastes;
separating the wastes into different portions for different downstream processes;
treating the wastes in an anaerobic digestion process for producing biogas;
applying a gasification process to the wastes for producing synthesis gas: and
applying an algal growth system for sequestering system-produced carbon dioxide (CO₂).

2. The method of claim 1, wherein receiving wastes comprises receiving at least one of municipal solid wastes, recyclable materials, ferrous metals, non-ferrous metals, organic materials, plastics, electronics, industrial waste, bio-hazardous materials, medical materials, hospital wastes, municipal waste water, municipal storm water, residential wastes, commercial wastes, wood residues, animal wastes, and food processing wastes.

3. The method of claim 1, wherein receiving wastes comprises receiving recyclable and non-recyclable materials, and
the method further comprising:
separating the recyclable materials from the non-recyclable materials; and
transporting the recyclable materials for recycling.

4. The method of claim 1, wherein receiving wastes comprises receiving the recyclable materials, and
the method further comprising applying gasification to the recyclable materials for producing a synthesis gas.

5. The method of claim 1, wherein receiving wastes comprises receiving organic materials, municipal wastewater and municipal storm water, and
the method further comprising processing the organic materials in an anaerobic digestion process.

6. The method of claim 1, wherein the biogas comprises H₂S, CO₂, and methane, and
the method further comprising:
removing at least a portion of the H₂S from the biogas;
applying an algal growth process to the biogas to remove and sequester at least a portion of the CO₂ therein to increase methane gas concentrations;
utilizing the methane gas as a fuel source to heat process water for treatment and steam production;
employing the heat and steam for electricity production;
employing the heat to process water for pasteurization; and
recovering heat energy for other system processes.

7. The method of claim 6, further comprising employing the heat for heating organic waste preparatory to the anaerobic digestion process.

8. The method of claim 1, wherein the gasification process produces slag and synthesis gas.

9. The method of claim 1, wherein applying a gasification process comprises utilizing one of a fluidized bed, a plasma arc, or a plasma torch.

10. The method of claim 1, further comprising:
using the synthesis gas in a power production system;
capturing carbon dioxide (CO₂) resulting from the power production system; and
applying the carbon dioxide to an algal system process for CO₂ sequestration.

11. The method of claim 1, further comprising using the synthesis gas in an integrated gasification combined cycle (IGCC) power production system for producing electricity.

12. The method of claim 1, further comprising utilizing unfiltered air produced in a tipping facility for use as air intake in a combustion system.

13. The method of claim 1, wherein applying a gasification process includes producing heat, and the method further comprises providing one of an Organic Rankine Cycle (ORC) system and Heat Recovery Steam Generator (HSRG).

14. The method of claim 1, further comprising providing a gas turbine power plant comprising:
a compressor for compressing air;
a combustor for burning fuel derived from system processes including at least one of gasification-derived synthesis gas, FT synthesis fuels, biogas, biodiesel, ethanol, other carbonaceous fuel with compressed air from the compressor to produce combustion gas;
a turbine configured to be driven by the combustion gas;
a generator configured to be driven by the turbine for producing electric power;
a heat exchanger configured to apply exhaust heat to a Rankine Cycle system for electricity production; and
a regenerative heat exchanger configured to heat the compressed air with the heat of exhaust gas of the turbine.

15. The method of claim 1, further comprising using the synthesis gas in a Fischer-Tropsch synthesis process for producing synthetic fuels.

16. The method of claim 1, further comprising utilizing a solar power system for producing electricity to implement one of more of the steps of claim 1.

17. The method of claim 1, further comprising:
sequestering waterborne waste from any of the steps of claim 1; and
using the water separated from waterborne waste for producing potable water.

18. The method of claim 1, wherein receiving wastes comprises receiving fibrous organic waste, grit material, and heavy waste material, and
the method further comprising:
separating the fibrous organic waste, grit material, and heavy waste material from each other;
preparing the fibrous organic waste for anaerobic digestion;
transporting the grit material for at least one of recycling and disposal; and
separating portions of the heavy waste material into recyclable ferrous metals and non-ferrous metals.

19. The method of claim 1, further comprising:
applying a gasification process to algae harvested from the algal growth system for producing additional synthesis gas;
using the additional synthesis gas in a power production system;
capturing carbon dioxide (CO₂) resulting from the power production system; and
applying the carbon dioxide to the algal growth system for CO₂ sequestration.

20. The method of claim 1, further comprising:
applying a transesterification/esterification process to lipids separated from harvested algae for producing biodiesel;
using the biodiesel in a power production system;
capturing carbon dioxide (CO₂) resulting from the power production system; and
applying the carbon dioxide to an algal system process for CO₂ sequestration.

21. The method of claim 1, further comprising:
applying a liquefaction/fermentation/distillation process to the algae for producing ethanol;
using the ethanol in a power production system;
capturing carbon dioxide (CO₂) resulting from the power production system; and
applying the carbon dioxide to the algal growth system for CO₂ sequestration.

22. The method of claim 1, further comprising:
dewatering effluent in the anaerobic digestion process; and
applying filtration and heat to removed water from dewatering for producing potable water.

23. The method of claim 1, further comprising:
dewatering effluent in the anaerobic digestion process for producing solids;
drying the solids;
applying a gasification process to the dried solids for producing a synthesis gas; and
applying vermiculture to the dried solids for producing a slow-release soil amendment.

24. A system for generating resources using wastes, the system comprising:
a tipping facility for separating the wastes into different portions for different downstream processes;
an anaerobic digester for treating the wastes for producing biogas;
a gasifier configured to use the wastes for producing synthesis gas; and
an algal growth system for sequestering system-produced carbon dioxide (CO₂).

25. The system of claim 24, wherein the wastes comprise recyclable materials, and wherein the gasifier is configured to use the recyclable materials for producing a synthesis gas.

26. The system of claim 24, wherein the wastes comprises organic materials, municipal wastewater and municipal storm water, and wherein the anaerobic digester is configured to process the organic materials.

27. The system of claim 24, wherein the biogas comprises H₂S, CO₂, and methane, and
wherein the algal growth process is configured to:
remove and sequester at least a portion of the CO₂ therein to increase methane gas concentrations; and
utilize the methane gas as a fuel source to heat process water for treatment and steam production.

28. The system of claim 24, wherein the gasifier produces slag and synthesis gas.

29. The system of claim 24, further comprising a power production system, and wherein the algal growth system applies carbon dioxide captured from the power production system for CO₂ sequestration.

30. The system of claim 24, further comprising an integrated gasification combined cycle (IGCC) power production system configured to use the synthesis gas for producing electricity.

31. The system of claim 24, further comprising a tipping facility for utilizing unfiltered air produced therein in a combustion system.

32. The system of claim 24, further comprising one of an Organic Rankine Cycle (ORC) system and Heat Recovery Steam Generator (HSRG).

33. The system of claim 24, further comprising a gas turbine power plant comprising:
a compressor for compressing air;
a combustor for burning fuel derived from system processes including at least one of gasification-derived synthesis gas, FT synthesis fuels, biogas, biodiesel, ethanol, other carbonaceous fuel with compressed air from the compressor to produce combustion gas;
a turbine configured to be driven by the combustion gas;
a generator configured to be driven by the turbine for producing electric power;
a heat exchanger configured to apply exhaust heat to a Rankine Cycle system for electricity production; and
a regenerative heat exchanger configured to heat the compressed air with the heat of exhaust gas of the turbine.

34. The system of claim 24, further comprising a Fischer-Tropsch synthesis configured to use the synthesis gas for producing synthetic fuels.

35. The system of claim 24, further comprising a solar power system for producing electricity to provide power to one of more of the anaerobic digester, the gasifier, and the algal growth system.

36. The system of claim 24, wherein the gasifier is configured to be applied to algae harvested from the algal growth system for producing additional synthesis gas,
wherein the system further comprises a power production system configured to utilize the additional synthesis gas and configured to produce carbon dioxide (CO₂), and
wherein the algal growth system uses the carbon dioxide for CO₂ sequestration.
